(19) United States
(12) Patent Application Publication
(10) Pub. No.: US 2021/0244743 A1
(43) Pub. Date: Aug. 12, 2021

(54) ANTI-VIRAL COMPOSITIONS AND METHODS OF USE

(71) Applicant: Al Therapeutics, Inc., Guilford, CT (US)

(72) Inventors: Sean Landrette, Meriden, CT (US); Peter R. Young, Guilford, CT (US); Henri Lichenstein, Guilford, CT (US); Murat Gunel, Guilford, CT (US)

(21) Appl. No.: 17/248,696

(22) Filed: Feb. 3, 2021

Related U.S. Application Data
(60) Provisional application No. 62/994,483, filed on Mar. 25, 2020.
(60) Provisional application No. 62/992,460, filed on Mar. 20, 2020.
(60) Provisional application No. 62/971,327, filed on Feb. 7, 2020.

(51) Int. Cl. A61K 31/5377; A61K 38/21; A61K 31/706; A61K 31/7052; A61K 31/4706; A61K 39/395; A61P 31/14

(52) U.S. Cl. CPC A61K 31/5377; A61K 38/21; A61K 31/706; A61P 31/14; A61K 31/4706; A61K 39/3955; A61K 31/7052

(57) ABSTRACT

The present invention relates to PIKfyve inhibitors, such as apilimod, and related compositions and methods for treating or preventing coronavirus infections.

### FIELD OF THE INVENTION

The present invention relates to anti-viral compositions comprising PIKfyve inhibitors and their use in treating coronavirus infections.

### BACKGROUND OF THE INVENTION

Many viruses enter the cell via endocytosis and utilize the endosomal network as a means to infiltrate the cell and replicate. For example, viral entry into cells may be mediated by a viral glycoprotein (GP), which attaches viral particles to the cell surface, delivers them to endosomes, and catalyzes fusion between viral and endosomal membranes. Rab9 GTPase was shown to be required for replication of HIV-1, filoviruses (such as Ebola and Marburg), and measles virus. Murray et al. 2005 J. Virology 79:11742-11751. Silencing Rab9 expression dramatically inhibited HIV replication, as did silencing the host genes encoding TIP47, p40, and PIKfyve, which also facilitate late-endosome-to-trans-Golgi vesicular transport. Reducing Rab9 expression also inhibited the replication of the enveloped Ebola and Marburg filoviruses and that of measles virus, but not the non-enveloped reoviruses. US 2007/0087008 (Hodge et al.) describes RAB9A, RAB11A, and modulators of those proteins as potentially useful for decreasing viral replication, especially HIV replication.

Coronaviruses are enveloped RNA viruses that cause respiratory, hepatic, and neurological disease (Weiss et al, (2011) Adv Virus Res 81:85-164; Cui et al., 2019 Nat Rev Microbiology 17:181-192). Recent outbreaks of severe acute respiratory syndrome (SARS) and Middle East respiratory syndrome (MERS) have revealed the potential for high pathogenicity (Cui et al., (2019) Nat Rev Microbiology 17:181-192). The high prevalence, wide distribution, genetic diversity, recombination, and frequent cross species infections of coronaviruses lend to the emergence of novel pathogenic strains (Cui et al., (2019) Nat Rev Microbiology 17:181-192; Wong et al., (2015) Cell Host and Microbe 18(4):398-401). Indeed, a novel pathogenic coronavirus, SARS-CoV-2, causing pneumonia and a high percentage of mortality is emerging in China (Zhu et al., 2020, N Engl J Med doi: 10.1056/NEJMoa2001017; Huang et al. (2020); The Lancet, doi.org/10.1016/S0140-6736(20)30183-5). The resulting disease has been named COVID-19. Attempts to treat SARS and MERS with approved antivirals and immunomodulators have proven ineffective in clinical trials (Zumla et al., (2016), Nat Rev Drug Discov. (2016) May; 15(5):327-47. PMID: 26868298).

More recently, preliminary data in vitro or from patients with the coronavirus SARS-CoV-2 has suggested promise for treatment with chloroquine, hydroxychloroquine, azithromycin and tocilizumab (Gautret et al., https://www.mediterranee-infection.com/wp-content/uploads/2020/03/Hydroxychloroquine_final_DOI_IJAA.pdf; Wang et al., Cell Res., (2020); Tocilizumab in COVID-19 Pneumonia NCT04317092, Phase 2 trial https://clinicaltrials.gov/). The treatment and prophylaxis of coronavirus infection remains an urgent unmet clinical need.

Additional studies indicate that the endo/lysosomal cholesterol transporter Niemann-Pick C1 (NPC1) acts as a post-endocytic intracellular receptor that is necessary for Ebola and Marburg virus penetration. Carette et al., (2011) Nature 477:340-343. Niemann-Pick C1 (NPC1) and the homotypic fusion and vacuole protein sorting (HOPS) complex were identified in a genome-wide haploid genetic screen as host factors for filovirus entry. The NPC1 locus was the single strongest hit, with 39 independent insertions. The HOPS complex was the next strongest hit. Additional genes whose products are involved in the biogenesis of endosomes (PIKfyve) and lysosomes (BLOC1S1, BLOC1S2), and in the targeting of luminal cargo to the endocytic pathway (GNPTAB) were also identified, but only NPC1 was validated in functional assays. For example, NPC1 function was required for infection by Ebola and Marburg viruses in human fibroblasts, NPC1 deficiency conferred resistance to viral infection in HAP1 and CHO cells, and NPC1 null mice were resistant to infection and pathogenesis of Ebola and Marburg viruses. WO 2012/103081 (Chandran et al.) describes methods for treating filovirus infection using an agent that inhibits, inter alia, NPC1 and the HOPS.

In yeast, fusion of the phagosome membrane to the lysosome membrane requires the HOPs complex and phosphatidylinositol 3,5-bisphosphate (PI(3,5)P₂). Synthesis of PI(3,5)P₂ is mediated by phosphatidylinositol-3-phosphate 5-kinase (PIKfyve). Phosphoinositides such as PI(3,5)P₂ are important lipid regulators of membrane trafficking and cellular signaling. Using the inhibitor YM201636, Jefferies et al. showed that inhibiting PIKfyve and blocking cellular production of PI(3,5)P₂ disrupts endomembrane transport and retroviral budding. Jefferies et al. EMBO rep. (2008) 9:164-170. Recently, the PIKfyve inhibitor apilimod has been reported to block filoviral entry (Nelson et al., (2017) PLoS Negl Trop Dis 11(4): e0005540; Qiu et al., (2018) Virology 513:17-28).

Cells of the innate immune system (neutrophils, natural killer cells, mast cells, dendritic cells, monocytes, macrophages, etc.) function to recognize infectious agents or endogenous malignancies and generate an inflammatory response. Cells of the adaptive immune system (B cells, αβ effector T cells and γδ effector T cells), proliferate and differentiate in response to the inflammatory environment created by the innate immune system. The adaptive immune system functions to directly destroy the pathogen or malignancy and in concert generate immunological memory to provide long-lasting protection against the specific pathogen.

The activation of resting T cells forms the basis of the adaptive immune response (Nathan (2013) Adv Physiol Education 37(4): 273-283). Activation involves the interaction of several molecules on the T cell including the T cell receptor (TCR), CD4/CD8, CD28, OX40, and 4-1BB co-stimulatory receptors with an antigen-presenting cell (APC) bearing an antigenic peptide from the pathogen or malignancy in the context of an appropriate class I or class II major histocompatibility complex (MHC), in addition to co-stimulatory molecules such as CD80/86, OX40 ligand and 4-1BB ligand. This T cell-APC interaction culminates in transcriptional changes within the T cell that result in the secretion of the pro-T cell proliferation factor IL-2.

Immune checkpoints exist to modulate the extent of the adaptive immune response to limit damages to host tissues. A subpopulation of regulatory T cells (Tregs) exists to suppress effector T cell proliferation and activity (Chaplin, (2010) J Allergy Clin Immunol February; 125(2 Suppl 2):S3-23). Furthermore, activated T cells express inhibitory receptors such as PD-1, which can function to limit co-stimulatory molecule ligation and signaling. PD-1 on the activated T-cell surface interacts with its ligands PD-L1 or PD-L2 on an APC or diseased cell, resulting in the dephosphorylation of TCR proximal kinases to limit TCR/CD28 signal transduction (Keir et al. (2008) Annual Review of Immunology 26: 677-704). PD-1/PD-L1 engagement within the tumor microenvironment or during chronic viral infection thereby results in impairment of the adaptive immune response by inducing T cell dysfunction through T-cell anergy, exhaustion and apoptosis, immunosuppressive IL-10 production, and enhancement of Treg differentiation as well as by mediating suppression of dendritic cell and cytotoxic T lymphocyte function (Chen et al. (2015) J Clin Invest. 125(9):3384-91).

Antagonizing PD-1/PD-L1 engagement significantly reduces tumor burden by enhancing the adaptive immune response in patients with advanced cancers (Topalian et al. (2012) The New England Journal of Medicine 366:2443-2454). Similarly, blocking PD-1/PD-L1 engagement during chronic viral infection restores function to exhausted T cells and leads to reductions in viral load in cases of chronic lymphocytic choriomeningitis virus, hepatitis C virus, human T cell lymphotrophic virus and human immunodeficiency virus (Barber et al. (2006) Nature 439: 682-687; Day et al. (2006) Nature 443: 350-354; Golden-Mason et al. (2007) Journal of Virology 81: 9249-9258; Yao et al. (2007) Viral Immunology 20: 276-287; and Kozako et al. (2009) Leukemia 23(2):375-82). High levels of PD-L1 expression on the cell surface of both tumor cells and virus-infected cells have been shown to directly inhibit T cell function and promote immune escape (Akhmetzyanova et al. (2015) PLoS Pathog 11(10); and Azuma T et al. (2008) Blood 111(7):3635-3643). Concordantly, high percentages of PD-1 expressing T cells were associated with higher viral loads, higher levels of inflammation and poor survival during the recent Ebola outbreak in West Africa, supporting the hypothesis that high PD-1/PD-L1 expression suppresses the adaptive immune response, thereby leading to poor viral clearance (Ruibal et al. (2016) Nature 533(7601):100-104; and Mohamadzadeh et al. (2007) Nat Rev Immunol; 7(7):556-67). Furthermore, an immune checkpoint blockade antibody has been demonstrated to reduce both tumor burden and viral load in hepatocellular carcinoma patients with HCV infection (Sangro et al. (2013) J Hepatol 59(1): 81-88).

Apilimod is an immunomodulatory small molecule that was first identified as an inhibitor of TLR-induced IL-12 and IL-23 cytokine production. IL-12 and IL-23 are produced by the innate immune system by APCs such as dendritic cells and macrophages. The secretion of IL-12 promotes the differentiation of helper T cells into IFNγ secreting T helper 1 (Th1) cells, thereby promoting inflammation and activation of the adaptive immune system (Teng et al. (2015) Nat Med. 21(7):719-29). While IL-23 can stabilize the Th17 response to maintain T cell activation, it can also suppress the innate immune response and promote tumorigenesis independent of the Th17 response (Teng et al. (2010) Proc Natl Acad Sci 4; 107(18):8328-33). Apilimod has been evaluated in the clinic for the inflammatory and auto-immune indications of Crohn's disease, psoriasis, and rheumatoid arthritis and has been shown to potently suppress the elevated T helper 1 (Th1) and Th17 responses that characterize such diseases, potentially through clearance of IL-12/23 producing CD11c+ dendritic cells (Cai et al. (2013) Chem Biol. 20(7):912-21; Krausz et al. (2012) Arthritis Rheum; 64(6):1750-5; Sands et al. (2010) Inflamm Bowel Dis; 16(7):1209-18; Wada et al. (2012) PLoS One; 7(4):e35069; and Wada et al. (2007) Blood 109, 1156-1164). In vitro, apilimod has been demonstrated to inhibit the production of a range of cytokines produced by stimulated human PBMCs with nanomolar potency, including IL-10, IL-6, IL-5, IL-4, and IFNγ, in addition to potently suppressing IL-12/23 production (Krausz et al. (2012) Arthritis Rheum; 64(6): 1750-5).

The present invention addresses the need for antiviral compositions and methods for the treatment of subjects infected with viruses and the prophylaxis of subjects who are at risk for viral infection, and particularly for human subjects infected with or at risk of infection with coronavirus.

### SUMMARY OF THE INVENTION

The present invention provides compositions and methods related to the use of PIKfyve inhibitors for the treatment and/or prophylaxis of coronavirus infections in a subject, preferably a human subject, in need of such treatment or prevention. In one aspect, the present invention is based upon the unexpected finding that apilimod inhibits the cytopathic effect of two coronaviruses, SARS and MERS, in an in vitro assay. In another aspect, the invention is based upon the anti-viral activity of apilimod alone against SARS-CoV-2 as well as its anti-viral activity in combination with remdesivir. Accordingly, the disclosure provides methods for the treatment and prevention of coronavirus infections using a PIKfyve inhibitor, preferably apilimod, or a pharmaceutically acceptable salt thereof, alone or in combination with one or more additional therapeutic agents selected from an antagonist of cellular PD-L1, PD-L2, or PD-1, an antiviral agent, and an anti-inflammatory agent.

In embodiments, the invention provides a method for treating or preventing a coronaviral infection in a subject in need thereof, the method comprising administering to the subject a composition comprising a therapeutically effective amount of at least one PIKfyve inhibitor, optionally in combination with an antagonist of cellular PD-L1, PD-L2, or PD-1, and further optionally in combination with an antiviral agent.

In embodiments, the PIKfyve inhibitor is selected from the group consisting of apilimod, APY0201, and YM-201636. In embodiments, the PIKfyve inhibitor is apilimod, or a pharmaceutically acceptable salt thereof. In embodiments, the apilimod is in the form of a free base or a dimesylate salt. In embodiments, the pharmaceutically acceptable salt is a monosalt selected from the group consisting of chloride, phosphate, maleate, L-tartrate, fumarate, DL lactate, and mesylate. In embodiments, the pharmaceutically acceptable salt is a disalt selected from the group consisting of mesylate, chloride, and bromide. Additional salt forms of apilimod are described infra.

In embodiments, the amount of apilimod administered to the subject is a prophylactically or therapeutically effective amount. In embodiments, the effective amount of apilimod free base, or a pharmaceutically acceptable salt thereof in humans is from about 70 to 1000 mg/day, from about 70 to 500 mg/day, from about 70 to 250 mg/day, from about 70 to 200 mg/day, from about 70 to 150 mg/day, of from about 70 to 100 mg/day.

In embodiments, the antagonist of cellular PD-L1, PD-L2, or PD-1 for use in combination therapy with apilimod as described herein is an antibody. In embodiments, the antibody is an anti-PD-L1 antibody selected from the group consisting of Tecentriq™ (atezolizumab), Avelumab (MSB0010718C) and Durvalumab (MEDI4736). In embodiments, the antibody is an anti-PD-1 antibody selected from the group consisting of Opdivo® (nivolumab) and Keytruda® (pembrolizumab). In embodiments, the prophylactically or therapeutically effective amount of the antagonist antibody in a human subject is from about 7 to 3500 mg/day, from about 70 to 1700 mg/day, from about 70 to 850 mg/day, from about 70 to 400 mg/day, from about 70 to 200 mg/day, of from about 70 to 150 mg/day. In embodiments, the daily amount is administered in a single day in a weekly or biweekly cycle, or in a 3 week cycle, or in a 4 week cycle, as described more fully infra.

In embodiments, the antagonist of cellular PD-L1, PD-L2, or PD-1 for use in combination therapy with apilimod as described herein is a small molecule. In embodiments, the prophylactically or therapeutically effective amount of the small molecule antagonist in a human subject is from about 70 to 1000 mg/day, from about 70 to 500 mg/day, from about 70 to 250 mg/day, from about 70 to 200 mg/day, from about 70 to 150 mg/day, of from about 70 to 100 mg/day.

In embodiments where the methods further comprise administering at least one additional anti-viral agent, the at least one additional anti-viral agent may comprise an antibody or a combination of antibodies, preferably human or humanized antibodies, but chimeric (e.g., mouse-human chimeras) antibodies are also acceptable. In embodiments, the at least one additional anti-viral agent comprises a recombinant protein or a combination of recombinant proteins. In embodiments, the at least one additional anti-viral agent comprises a small interfering RNA (siRNA) or a combination of siRNA molecules. In embodiments, the antibody, recombinant protein, siRNA, or combination of any of the foregoing targets one or more coronavirus proteins. In embodiments, the one or more coronavirus proteins is selected from the group consisting of a polymerase, a membrane-associated protein, a polymerase complex protein. a protease, a helicase, an envelope, a nucleocapsid, a spike glycoprotein, a viral structural, or a viral accessory protein. In embodiments, the siRNA or combination of siRNAs, the antibody or combination of antibodies, the protein or combination of proteins targets one or more host proteins. In embodiments, the one or more host proteins is selected from the group consisting of an interferon, a cell surface receptor, a protease, or a protein involved in endosomal trafficking or acidification. In embodiments, the antibody, recombinant protein, siRNA, or combination of any of the foregoing target all of these proteins.

In embodiments, the anti-viral agent is selected from one or more of an interferon, remdesivir, azithromycin, hydroxychloroquine and chloroquine. In embodiments, the methods comprise administering the apilimod and anti-viral agent in combination with an anti-inflammatory agent. In embodiments, the anti-inflammatory agent is selected from tocilizumab and sarilumab. In embodiments, the apilimod is administered in a separate dosage form from the anti-viral agent and the anti-inflammatory agent.

In embodiments, the subject in need is one who has symptoms of a respiratory viral infection including one or more of sore throat, nasal congestion and/or discharge, shortness of breath, difficulty breathing, and fever.

In embodiments, the coronavirus is SARS-CoV-2. In embodiments, the coronavirus is SARS-CoV-2 and the methods comprise administering one or more additional therapeutic agents selected from remdesivir, azithromycin, hydroxychloroquine, chloroquine, tocilizumab and sarilumab. In embodiments, the apilimod is administered in a separate dosage form, or in the same dosage form, as the one or more additional therapeutic agents.

In the methods described here, the at least one PIKfyve inhibitor can be administered by any suitable route. In embodiments, administration is via an oral, intravenous, or subcutaneous route. In embodiments, administration is once daily, twice daily, or continuous for a period of time, for example one or several days or one or several weeks. Continuous administration may be performed, for example, by using a slow release dosage form that is e.g., implanted in the subject, or via continuous infusion, for example using a pump device, which also may be implanted.

In embodiments, the at least one PIKfyve inhibitor is apilimod or a pharmaceutically acceptable salt thereof and the apilimod is administered in an amount of 70 to 1000 mg/day. In one embodiment, administration is effective to achieve a plasma concentration of apilimod in the subject in the range of from 50 to 1000 nM.

The invention also provides a pharmaceutical pack or kit comprising, in separate containers or in a single container, a unit dose of apilimod, optionally a unit dose of an antagonist of cellular PD-L1, PD-L2, or PD-1, and further optionally at least one additional anti-viral agent. In embodiments, the pharmaceutical pack or kit comprises at least one PIKfyve inhibitor that is an apilimod composition selected from apilimod free base, or any pharmaceutically acceptable salt of apilimod, or a racemically pure enantiomer of an active metabolite of apilimod, and combinations thereof. In embodiments, the pharmaceutical pack or kit comprises a PD-L1 antagonist and/or a PD-L2 antagonist and/or a PD-1 antagonist selected from the group consisting of an antibody or fragment thereof, peptides, polypeptides or fragments thereof, small molecules, and inhibitory nucleic acids. In embodiments, the pharmaceutical pack or kit comprises a PD-L1 antagonist. In one embodiment, the PD-L1 antagonist is an antibody. In one embodiment, the PD-L1 antagonist is a monoclonal antibody.

In embodiments, the pharmaceutical pack or kit comprises, in separate containers or in a single container, a unit dose of apilimod, or a pharmaceutically acceptable salt thereof, and a unit dose of one or more additional therapeutic agents selected from remdesivir, azithromycin, hydroxychloroquine, chloroquine, tocilizumab and sarilumab.

### BRIEF DESCRIPTION OF THE DRAWINGS

FIG. 1: Apilimod displays antiviral activity against SARS and MERS viruses in vitro. Average EC₅₀ of apilimod in the neutral red cytopathic effect antiviral assay from independent experiments (n=3 SARS, n=2 MERS).

FIG. 2A-F: Apilimod inhibits SARS-CoV-2 infection. (A) Vero-E6 cells were treated with SARS-CoV-2 and 3 hours later with varying doses of apilimod dimesylate and assayed 48 hours later for viral transcript copy number. Dose-response curves are shown. An EC50 of 2.2 uM was calculated. (B) Vero cells were treated with varying doses of apilimod dimesylate without viral challenge and cytotoxicity was measured. An CC50 of 20 uM was calculated. (C) Vero cells were infected with SARS-CoV-2 and 3 hours later treated with varying doses of remdesivir and assayed 48 hours later for viral transcript copy number. An EC50 of 0.7 uM was calculated. (D) Vero cells were treated with varying doses of remdesivir without viral challenge and cytotoxicity was measured. An CC50 of 205 uM was calculated. (E) Remdesivir at 0.5 uM was tested as single agent and in combination with 10, 3, or 1 uM apilimod dimesylate 3 hours after addition of virus. Single dose apilimod dimesylate (10, 3, or 1 uM) and DMSO controls were also assessed. Percent inhibition is displayed, single agent=light gray; combination with remesivir=dark gray. Significance determined with One-way ANOVA, Tukey's multiple comparison test and is displayed versus remdesivir (0.5 uM)/versus single agent apilimod (****P<0.0001, ***P<0.001, **P<0.01, *P<0.05) (F) Fold reduction in viral transcript from E is plotted, single agent=light gray; combination with remesivir=dark gray. Significance determined on log transformed data with Tukey's multiple comparison test and is displayed versus remdesivir (0.5 uM)/versus single agent apilimod (****P<0.0001, ***P<0.001, **P<0.01).

### DETAILED DESCRIPTION OF THE INVENTION

The present invention provides compositions and methods related to the use of PIKfyve inhibitors for treating or preventing a coronavirus infection in a subject, preferably a human subject, in need of such treatment or prevention.

In embodiments, the invention provides methods for the treatment of coronavirus infections in a subject by administering to the subject a therapeutically effective amount of a PIKfyve inhibitor. In embodiments, the invention provides pharmaceutical compositions comprising a therapeutically effective amount of a PIKfyve inhibitor. In embodiments, the PIKfyve inhibitor is selected from the group consisting of apilimod, APY0201, YM201636, and pharmaceutically acceptable salts thereof.

In embodiments, the invention provides pharmaceutical compositions comprising a therapeutically effective amount of at least one PIKfyve inhibitor and a therapeutically effective amount of an antagonist of cellular PD-L1, PD-L2, or PD-1.

In embodiments, the antagonist of cellular PD-L1, PD-L2, or PD-1 is a PD-L1 antagonist. Programmed death-ligand 1 (PD-L1) “Protein PD-L1”, “PD-L1”, “PDL1”, “PDCDL1”, “hPD-L1”, “hPD-L1”, “CD274” and “B7-H1” are used interchangeably, and include variants, isoforms, species homologs of human PD-L1, and analogs having at least one common epitope with PD-L1. Programmed death-ligand 1 (PD-L1) is a transmembrane protein that plays a role in suppressing the immune system during particular events such as pregnancy, tissue allografts, autoimmune disease and other disease states such as hepatitis. The complete PD-L1 sequence can be found under GENBANK® Accession No. NP 054862.

In embodiments, the antagonist of cellular PD-L1, PD-L2, or PD-1 is a PD-L2 antagonist. Programmed cell death 1 ligand 2 (also known as PD-L2, B7-DC) is a protein that in humans is encoded by the PDCD1LG2 gene. The complete PD-L2 sequence can be found under GENBANK® Accession No. Q9BQ51.2.

In embodiments, the antagonist of cellular PD-L1, PD-L2, or PD-1 is a PD-1 antagonist. “PD-1” refers to programmed death-1 receptor. The terms “Programmed Death 1”, “Programmed Cell Death 1”, “Protein PD-1”, “PD-1”, “PD1”, “PDCD1”, “hPD-1” and “hPD-I” are used interchangeably, and include variants, isoforms, species homologs of human PD-1, and analogs having at least one common epitope with PD-1. PD-1 is a member of the extended cluster of differentiation 28(CD28)/cytotoxic T-lymphocyte-associated protein 4 (CTLA-4) family of T cell regulators. The complete PD-1 sequence can be found under GENBANK® Accession No. U64863. The complete CTLA-4 sequence can be found under GENBANK® Accession No. P16410.3. PD-1 signaling refers to a negative co-stimulatory signal regulating T cell activation provided by PD-1 and its binding partner, PD-L1. PD-1 can be expressed on T cells, B cells, natural killer T cells, activated monocytes and dendritic cells (DCs). PD-1 signaling typically has a greater effect on cytokine production than on cellular proliferation, with significant effects on IFN-γ, TNF and IL-2 production. PD-1 binds two ligands, PD-L1 and PD-L2. Inhibitors blocking the PD-L1:PD-1 interaction are known from, for example, WO2001014557, WO2002086083, WO2007005874, WO2010036959, WO2010077634 and WO2011066389.

As used herein, an “antagonist” may refer to an antibody or fragment thereof, peptides, polypeptide or fragments thereof, small molecules, and inhibitory nucleic acids or fragments thereof that interferes with the activity or binding of another, for example, by competing for the one or more binding sites of an agonist, but does not induce an active response.

An “antagonist antibody” or a “blocking antibody” is one that inhibits or reduces a biological activity of the antigen it binds to. In some embodiments, blocking antibodies or antagonist antibodies substantially or completely inhibit the biological activity of the antigen. The anti-PD-L1 antibodies and the anti-PD-L2 antibodies of the invention block the interaction with its receptor PD-1, and thus the signaling through PD-1. The anti-PD1 antibodies of the invention block the receptor. Alternatively, an “agonist” or activating antibody is one that enhances or initiates signaling by the antigen to which it binds. In some embodiments, agonist antibodies cause or activate signaling without the presence of the natural ligand.

In embodiments, the antagonist (i.e., the PD-L1 antagonist and/or a PD-L2 antagonist and/or PD-1 antagonist) is a PD-L1 antibody. Exemplary PD-L1 antibodies may include antibodies purchased from any suitable distributor, including, for example, Abcam, BD Biosciences, BioRad, Cell Signaling, EMD Millipore, Novus Biologicals, R&D Systems, and the like. For example, exemplary PD-L1 antibodies from Abcam may include, but are not limited to: ab205921 (rabbit monoclonal), ab58810 (rabbit polyclonal), ab209960 (rabbit monoclonal), ab210931 (mouse monoclonal), ab209959 (rabbit monoclonal), ab209889 (rabbit monoclonal), ab209961 (rabbit monoclonal), ab180370 (mouse monoclonal), ab109052 (mouse monoclonal), or ab80391 (mouse monoclonal).

In embodiments, the antagonist is a PD-L1 antibody selected from the group consisting of Tecentriq™ (atezolizumab), Avelumab (MSB0010718C) and Durvalumab (MEDI4736).

In embodiments, the antagonist is a PD-L2 antibody. Exemplary PD-L2 antibodies may include antibodies purchased from any suitable distributor, including, for example, R&D Systems, EMD Millipore, Novus Biologicals, Cell Signaling, and the like. Exemplary PD-L2 antibodies may include, but are not limited to, antibodies purchased from R&D Systems (Item No. MAB1224-100; mouse monoclonal or Item No. AF1224-SP; goat polyclonal or Item No. BAF1224; goat polyclonal), Novus Biologicals (Item No. NBP1-76770; rabbit polyclonal), or EMD Millipore (Item No. ABC327; rabbit polyclonal).

In embodiments, the antagonist is a PD-1 antibody. In embodiments, the antagonist is a PD-1 antibody selected from the group consisting of Opdivo® (nivolumab) and Keytruda® (pembrolizumab).

In embodiments, the antagonist is a CTLA-4 antibody, e.g., Yervoy® (ipilimumab).

By “antigen” is defined as a molecule that provokes an immune response. This immune response may involve either antibody production, or the activation of specific immunologically-competent cells, or both. For example, any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein.

By “small molecule” it may be referred to broadly as an organic, inorganic or organometallic compound with a low molecular weight compound (e.g., a molecular weight of less than about 1,000 Da). The small molecule may have a molecular weight of less than about 1,000 Da, or a molecular weight of less than about 9000 Da, molecular weight of less than about 800 Da, molecular weight of less than about 700 Da, molecular weight of less than about 600 Da, molecular weight of less than about 500 Da, molecular weight of less than about 400 Da, molecular weight of less than about 300 Da, molecular weight of less than about 200 Da, molecular weight of less than about 100 Da, molecular weight of less than about 50 Da.

In embodiments, the antagonist (i.e., the PD-L1 antagonist and/or a PD-L2 antagonist and/or PD-1 antagonist) is a small molecule. In an embodiment, the small molecule is AUPM-170 (alternatively named CA-170, Curis).

As used herein, “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

Inhibitory nucleic acid, siRNA may refer to a double stranded RNA. Optimally, an siRNA is 18, 19, 20, 21, 22, 23 or 24 nucleotides in length and has a 2-base overhang at its 3′ end. These dsRNAs can be introduced to an individual cell or to a whole animal; for example, they may be introduced systemically via the bloodstream. Such siRNAs are used to downregulate mRNA levels or promoter activity.

In embodiments, the viral infection is caused by a coronavirus selected from the group consisting of novel Coronavirus (SARS-CoV-2), severe acute respiratory system virus (SARS-CoV), middle east respiratory syndrome virus (MERS-CoV), alpha coronavirus 229E, alpha coronavirus NL63, beta coronavirus OC43, and beta coronavirus HKU1. In embodiments, the PIKfyve inhibitor is apilimod. Apilimod is a selective inhibitor of PIKfyve (Cai et al. 2013 Chem. & Biol. 20:912-921). Based upon its ability to inhibit IL-12/23 production, apilimod has been suggested as useful for treating inflammatory and autoimmune diseases such as rheumatoid arthritis, sepsis, Crohn's disease, multiple sclerosis, psoriasis, or insulin dependent diabetes mellitus, and in cancers where these cytokines were believed to play a pro-proliferative role. In accordance with the methods described here, the inventors have found that apilimod has coronavirus antiviral activity, including against SARS and MERS. This was unexpected from previous work demonstrating that apilimod inhibited filoviral entry (e.g., Ebola and Marburg viruses) because host factors necessary for viral entry are different between distinct viral families and can differ even within a single family. This has been shown for coronaviruses in which different strains utilize different host proteases and receptors (Totura et al, (2019) Expert Opin Drug Discov 14(4):397-412). Accordingly, the methods described here provide an alternative antiviral therapy as compared to conventional approaches such as vaccines and monoclonal antibodies targeting specific viral proteins, which have proven to be ineffective against diverse coronavirus pathogens. See e.g., Totura et al, (2019) Expert Opin Drug Discov 14(4):397-412).

As used herein, the term “apilimod” refers to apilimod free base having the structure shown in Formula I:

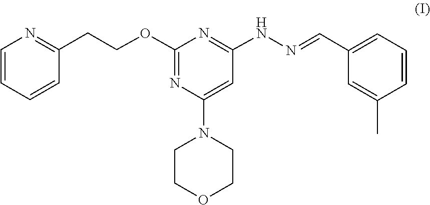 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0.

Apilimod can be prepared, for example, according to the methods described in U.S. Pat. Nos. 7,923,557, and 7,863,270, and WO 2006/128129.

In embodiments, a pharmaceutically acceptable salt form of apilimod may be used in the methods and compositions described here. In embodiments, the apilimod may be apilimod dimesylate.

In embodiments, the apilimod may be administered in combination with at least one additional PIKfyve inhibitor selected from APY0201 and YM-201636.

The chemical name of APY0201 is (E)-4-(5-(2-(3-methylbenzylidine)hydrazinlyl)-2-(pyridine-4-yl)pyrazolol[1,5-a]pyrimidin-7-yl)morpholine. APY0201 is a selective PIKfyve inhibitor (Hayakawa et al. (2014) Bioorg. Med. Chem. 22:3021-29). APY0201 directly interacts with the ATP-binding site of PIKfyve kinase, which leads to suppression of PI(3,5)P2 synthesis, which in turn suppresses the production of IL-12/23.

The chemical name for YM201636 is 6-amino-N-(3-(4-morpholinopyrido[3′,2′:4,5]furo[3,2-d]pyrimidin-2-yl)phenyl)nicotinamide (CAS number is 371942-69-7). YM201636 is a selective inhibitor of PIKfyve (Jefferies et al. EMBO rep. (2008) 9:164-170). It reversibly impairs endosomal trafficking in NIH3T3 cells, mimicking the effect produced by depleting PIKfyve with siRNA. YM201636 also blocks retroviral exit by budding from cells, apparently by interfering with the endosomal sorting complex required for transport (ESCRT) machinery. In adipocytes, YM-201636 also inhibits basal and insulin-activated 2-deoxyglucose uptake (IC₅₀=54 nM).

As used herein, the term “pharmaceutically acceptable salt,” is a salt formed from, for example, an acid and a basic group of a compound, particularly a PIKfyve inhibitor as described herein. Illustrative salts include, but are not limited to, sulfate, citrate, acetate, oxalate, chloride, bromide, iodide, nitrate, bisulfate, phosphate, acid phosphate, isonicotinate, L-lactate, D-lactate, DL-lactate, salicylate, acid citrate, L-tartrate, D-tartrate, DL-tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate (mesylate), ethanesulfonate, benzenesulfonate, p-toluenesulfonate, and pamoate (e.g., 1,1′-methylene-bis-(2-hydroxy-3-naphthoate)) salts. In embodiments, the pharmaceutically acceptable salt form of apilimod is a methanesulfonate salt form, alternatively referred to as a mesylate salt.

The term “pharmaceutically acceptable salt” also refers to a salt prepared from a compound having an acidic functional group, such as a carboxylic acid functional group, and a pharmaceutically acceptable inorganic or organic base.

The term “pharmaceutically acceptable salt” also refers to a salt prepared from a compound having a basic functional group, such as an amino functional group, and a pharmaceutically acceptable inorganic or organic acid.

In embodiments, the salts of apilimod include disalts in which the Bronsted acid is selected from a group consisting of hydrochloric acid, hydrobromic acid, nitric acid, sulfuric acid, hydrobromic acid, hydroiodic acid, perchloric acid, methanesulfonic acid, phorphoric acid, alkylsulfonic acids, arylsulfonic acids, halogenated alkylsulfonic acids, halogentated arylsulfonic acids, halogenated alkylsulfonic acids, halogenated acetic acids, picric acid, oxalic acid, citric acid, formic acid, ascorbic acid, benzoic acid and other salts possessing sufficient acidity to form a crystalline disalt of apilimod. In an embodiment, the salt form of apilimod is a dimesylate.

The salts of the compounds described herein can be synthesized from the parent compound by conventional chemical methods such as methods described in Pharmaceutical Salts: Properties, Selection, and Use, P. Hemrich Stahl (Editor), Camille G. Wermuth (Editor), ISBN: 3-90639-026-8, August 2002. Generally, such salts can be prepared by reacting the parent compound with the appropriate acid in water or in an organic solvent, or in a mixture of the two.

One salt form of a compound described herein can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid using routine methods.

### Methods of Treatment

The present invention provides methods for the treatment of coronaviral infections in a subject in need thereof by administering to the subject a therapeutically effective amount of at least one PIKfyve inhibitor. In embodiments, the at least one PIKfyve inhibitor is selected from apilimod, APY0201, YM-201636, and pharmaceutically acceptable salts thereof.

In some embodiments, the PIKfyve inhibitor is administered in combination with a PD-L1 antagonist and/or a PD-L2 antagonist and/or PD-1 antagonist.

The present invention further provides the use of at least one PIKfyve inhibitor, either alone or in combination with, a PD-L1 antagonist and/or a PD-L2 antagonist and/or a PD-1 antagonist for the preparation of a medicament useful for the treatment of viral infections.

The term “therapeutically effective amount” refers to an amount sufficient to treat, ameliorate a symptom of, reduce the severity of, or reduce the duration of a viral infection, or enhance or improve the therapeutic effect of another therapy, e.g., another antiviral therapy, when administered in combination with a PIKfyve inhibitor or as part of a therapeutic regimen that includes administering a PIKfyve inhibitor, either alone or in combination with a PD-L1 antagonist and/or a PD-L2 antagonist and/or a PD-1 antagonist as described herein.

In embodiments, the therapeutically effective amount is an amount effective to achieve one or more of the following: inhibit cellular PIKfyve activity, substantially prevent viral entry into a subject's cells, reduce the amount of viral particles which gain entry to a subject's cells, reduce viral replication within the subject's cells, ameliorate one or more symptoms associated with viral infection of the subject, and reduce the severity of one or more symptoms associated with viral infection of the subject.

In embodiments, the therapeutically effective amount is in an amount to enhance host defense against viral pathogens. In embodiments, the therapeutically effective amount is in an amount that is synergistic to promote an immune-activating environment that enhances host defense against viral pathogens.

In embodiments, the therapeutically effective amount is an amount sufficient to reduce the magnitude of, or prevent the onset of, a cytokine storm in the subject.

In embodiments, the therapeutically effective amount is an amount sufficient to reduce viral load. In embodiments, the viral load is reduced by 5% or greater, 10% or greater, 20% or greater, 30% or greater, 40% or greater, 50% or greater, or 75% or greater. In embodiments, the viral load is reduced by at least 0.5 log unit, at least 1 log unit, at least 2 log units, at least 3 log units, at least 4 log units, at least 10 log units, at least 15 log units, or by at least 20 log units.

A therapeutically effective amount can range from about 0.001 mg/kg to about 1000 mg/kg, more preferably 0.01 mg/kg to about 100 mg/kg, more preferably 0.1 mg/kg to about 10 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents. See, e.g., U.S. Pat. No. 7,863,270, incorporated herein by reference.

In embodiments, the therapeutically effective amount of apilimod in humans is from about 70 to 1000 mg/day, from about 70 to 500 mg/day, from about 70 to 250 mg/day, from about 70 to 200 mg/day, from about 70 to 150 mg/day, of from about 70 to 100 mg/day.

In embodiments, the PD-L1/L2 or PD-1 antagonist is an antibody and the antibody is administered at a dosage regimen from about 7 to 3500 mg/day, from about 70 to 1700 mg/day, from about 70 to 850 mg/day, from about 70 to 400 mg/day, from about 70 to 200 mg/day, of from about 70 to 150 mg/day once a week, or once every 2 weeks, or once every 3 weeks, or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks). In embodiments, the antagonist is administered at a dosage regimen of 70-1000 mg/day for 2, 4, 12, or 16 weeks. Alternatively or subsequently, the antagonist is administered at a dosage regimen of 7 mg-3500 mg twice a day for 4 weeks, 8 weeks, 12 weeks, 16 weeks, or longer.

In embodiments, the PD-L1/L2 or PD-1 antagonist is a small molecule and the small molecule is administered at a dosage regimen of 70-1000 mg/day (e.g., 70, 75, 80, 85, 90, 95, 100, 125, 125, 150, 175, 200, 225, 250, 275, 300, 350, 400, 450, 500, 550, 600, 650, 700, 750, 800, 850, 900, 950, or 1000 mg/day) for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks). In embodiments, the small molecule antagonist is administered at a dosage regimen of 70-1000 mg/day for 2, 4, 12, or 16 weeks. Alternatively or subsequently, the small molecule antagonist is administered at a dosage regimen of 35 mg-500 mg twice a day for 4 weeks, 8 weeks, 12 weeks, 16 weeks, or longer.

In some embodiments, the methods comprise administering the PIKfyve inhibitor in combination with a PD-L1/L2 or PD-1 antagonist and an optional anti-viral agent according to a specified dosing schedule or therapeutic regimen. For example, the PIKfyve inhibitor can be administered once daily or from two to five times daily. In embodiments, apilimod, APY0201, or YM-201636, either alone or in combination with a PD-L1/L2 or PD-1 antagonist and/or anti-viral agent is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In the context of combination therapy, the PIKfyve inhibitor and the PD-L1/L2 or PD-1 antagonist may be administered in separate dosage forms, or in the same dosage form. Where the inhibitor and the antagonist are administered in separate dosage forms, they may be administered at the same time, or at different times. For example, the inhibitor and/or antagonist may be administered thrice daily, twice daily, once daily, or in a defined cycle of, e.g., fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks. In embodiments where the antagonist is an antibody, the antibody will generally be administered only once a day, and generally on a single day once a week, or once every 2 weeks, or once every 3 weeks, or once every 4 weeks.

In accordance with the methods described herein, a “subject in need of” is a subject having a coronavirus infection, or a subject having an increased risk of developing a coronavirus infection relative to the population at large. The subject in need thereof can be one that is “non-responsive” or “refractory” to a currently available therapy for the viral disease. In this context, the terms “non-responsive” and “refractory” refer to the subject's response to therapy as not clinically adequate to relieve one or more symptoms associated with the viral infection. In one aspect of the methods described here, the subject in need thereof is a subject having a viral disease caused by a coronavirus who is refractory to standard therapy.

A “subject” includes a mammal. The mammal can be e.g., any mammal, e.g., a human, primate, mouse, rat, dog, cat, cow, horse, goat, camel, sheep or a pig. Preferably, the mammal is a human. The term “patient” refers to a human subject.

As used herein, “treatment”, “treating” or “treat” describes the management and care of a patient for the purpose of combating a viral disease and includes the administration of a PIKfyve inhibitor, preferably apilimod, either alone or in combination with a PD-L1 antagonist and/or a PD-L2 antagonist and/or a PD-1 antagonist to alleviate the symptoms or complications of the viral disease.

As used herein, “prevention,” “preventing” or “prevent” describes reducing or eliminating the onset of the symptoms or complications of the viral disease, includes the administration of a PIKfyve inhibitor, preferably an apilimod composition, either alone or in combination with a PD-L1 antagonist and/or a PD-L2 antagonist and/or a PD-1 antagonist to reduce the onset, development or recurrence of symptoms of the viral disease.

The present invention also provides methods comprising combination therapy. As used herein, “combination therapy” or “co-therapy” includes the administration of a therapeutically effective amount of a PIKfyve inhibitor, preferably apilimod, either alone or in combination with an antagonist of PD-L1, PD-L2, or PD-1, as part of a specific treatment regimen intended to provide a beneficial effect from the co-action of the active agents in the regimen.

“Combination therapy” is not intended to encompass the administration of two or more therapeutic compounds as part of separate monotherapy regimens that incidentally and arbitrarily result in a beneficial effect that was not intended or predicted.

Thus, the invention provides methods of treating a subject for a viral disease or viral infection (the terms “viral disease” and “viral infection” are used interchangeably herein) using a combination therapy comprising a PIKfyve inhibitor, preferably apilimod, alone or in combination with an antagonist of PD-L1, PD-L2, or PD-1, in an anti-viral regimen for the treatment of the viral disease.

In embodiments, the combination therapy may comprise a PIKfyve inhibitor administered in combination with an antiviral agent. In some embodiments, the antiviral agent is selected from an anti-viral vaccine, a nucleotide analogue, a cytokine (e.g., an interferon), an immunoglobulin, and combinations thereof. In embodiments, the antiviral agent is selected from an inhibitor of one or more of NPCI, VPSII, VPSI6, VPSI8, Vacuolar Protein Sorting 33 Homolog A (VPS33A), Vacuolar Protein Sorting 39 Homolog (VPS39), Vacuolar Protein Sorting 41 Homolog (VPS41), BLOCISI, BLOCIS2, N-Acetylglucosamine-1-Phosphate Transferase, Alpha And Beta Subunits (GNPT-AB), Phosphoinositide Kinase, FYVE Finger Containing (PIKFYVE), ARGHGAP23, coat protein complex 1 (COPI), coat protein complex II (COPII), Mannose-6-phosphate receptor binding protein 1 (TIP47), Interleukin 12 (IL-12 or P40), Rab GTP-binding proteins (e.g., Rab9), clathrin, activator protein 1 (AP1), adaptor protein 3 (AP3), vesicle soluble N-ethylmaleimide-sensitive factor attachment protein receptor (v-SNARE), target soluble N-ethylmaleimide-sensitive factor attachment protein receptor (t-SNARE), ADP-ribosylation factor 1 (ARFs), Ras GTP-ases, and a combinations thereof.

Further non-limiting examples of anti-viral agents that may be used in combination with a PIKfyve inhibitor as described herein include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Chloroquine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Remdesivir; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

In certain embodiments the at least one PIKfyve inhibitor is provided in a single dosage form in combination with one or more antiviral agents. In embodiments, the PIKfyve inhibitor is apilimod.

In embodiments, the at least one PIKfyve inhibitor is provided in a separate dosage form from the one or more additional agents. Separate dosage forms are desirable, for example, in the context of a combination therapy in which the therapeutic regimen calls for administration of different therapeutic agents at different frequencies or under different conditions, or via different routes.

In embodiments, administration of the at least one PIKfyve inhibitor as described herein is accomplished via an oral dosage form suitable for oral administration. In another embodiment, administration is by an indwelling catheter, a pump, such as an osmotic minipump, or a sustained release composition that is, for example, implanted in the subject.

### Pharmaceutical Compositions and Formulations

The disclosure provides pharmaceutical compositions comprising an effective amount of at least one PIKfyve inhibitor and at least one pharmaceutically acceptable excipient or carrier, wherein the effective amount is as described above in connection with the methods of the invention.

In embodiments, the PIKfyve inhibitor is selected from apilimod, APY0201, YM-201636, and pharmaceutically acceptable salts thereof. In embodiments, the PIKfyve inhibitor is apilimod, or a pharmaceutically acceptable salt thereof.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating viral infections are described above.

A dose may be provided in unit dosage form. For example, the unit dosage form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g., liquids, aerosols, solutions, inhalants, mists, sprays; or solids, powders, ointments, pastes, creams, lotions, gels, patches and the like) for administration by any desired route (e.g., pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present invention may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the invention are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

All percentages and ratios used herein, unless otherwise indicated, are by weight. Other features and advantages of the present invention are apparent from the different examples. The following examples illustrate different components and methodology useful in practicing the present invention. The examples do not limit the claimed invention. Based on the present disclosure the skilled artisan can identify and employ other components and methodology useful for practicing the present invention.

### EXAMPLES

### Example 1

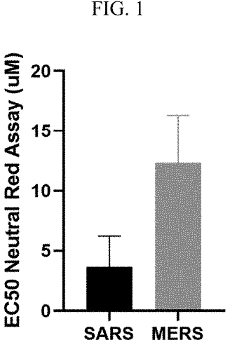

We conducted an in vitro antiviral activity assay (cytopathic effect reduction) to test apilimod against coronaviruses. Briefly, Vero cells were treated with 8 half-log, serial dilutions of apilimod dimesylate (52 nM-164 uM) in triplicate in MEM medium with 2% FBS and 50 mg/mL gentamicin and infected with SARS or MERS. Virus only and media only controls were also included. The cells were incubated at 37° C.+5% CO₂ until a cytopathic effect was observed microscopically (3-5 days). Cells were then stained with 0.011% neutral red dye for approximately 2 hours, washed and then incubated for 30 minutes with an equal volume of sorensen citrate buffer/ethanol. The absorbance was read on a spectrophotometer at 540 nm and was converted to percent of cell control, normalizing to virus only controls, and regression analysis was used to calculate the 50% virus inhibitory concentration (EC₅₀). FIG. 1 shows that the average EC₅₀ from independent experiments was 3.7 uM for SARS (n=3) and 13.8 uM for MERS (n=2).

These results indicated, unexpectedly, that apilimod exhibits antiviral activity against coronaviruses such as SARS and MERS. This was unexpected from the previously documented ability of apilimod to inhibit viral entry of filoviruses such as Ebola and Marburg at least because the host factors necessary for viral entry are generally different among different viral families and can be divergent even within a single family, as has been shown for coronaviruses in which different strains utilize different host proteases and receptors (Totura et al, (2019) Expert Opin Drug Discov 14(4):397-412).

As conventional approaches like vaccines and monoclonal antibodies that target viral proteins are ineffective against diverse coronavirus pathogens and resistance mutations quickly develop (Totura et al, (2019) Expert Opin Drug Discov 14(4):397-412), the discovery of a broad acting anti-viral drug is not easily identifiable or obvious but would be immensely valuable to society. Indeed, the SARs virus can tolerate mutations in multiple epitopes that not only confer resistance, but enhance the pathogenesis of the virus as demonstrated in animal models (Sui et al. (2014) J Virol. 88(23):13769-80). In addition, targeting the viral polymerase and proofreading exonuclease with nucleoside analogues can select for the emergence of resistant strains (Agostini et al. (2018) mBio 9(2) pii: e00221-18).

### Example 2: Apilimod Inhibits SARS-CoV-2 Infection

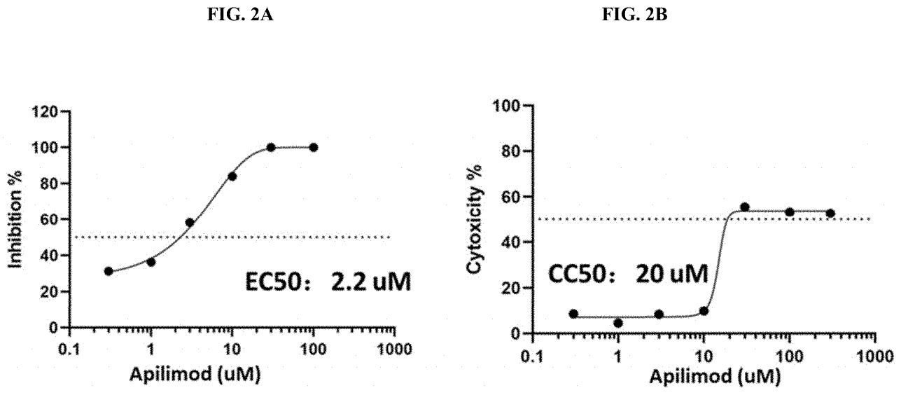

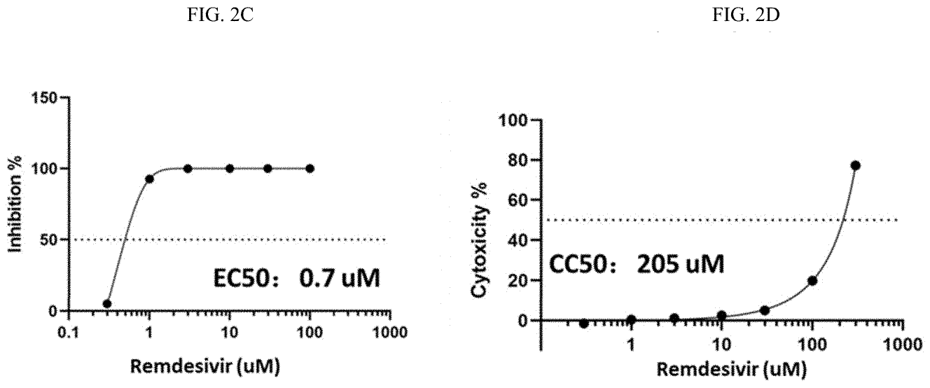

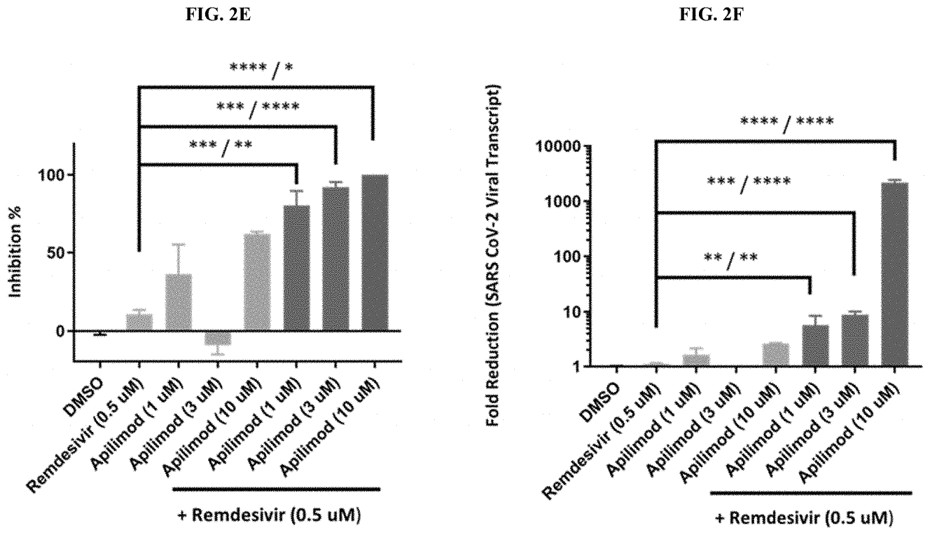

The antiviral activity of apilimod dimesylate was tested against a clinical isolate of SARS-CoV-2 in Vero E6 cells. The cells were infected with a 100 TCID50 dose for 3 hours before removing the virus and adding various doses of apilimod or remdesivir. Forty-eight hours later, RNA was extracted from the cell supernatants and viral transcript levels were measured via quantitative real-time PCR. The percent of viral inhibition was calculated based on comparison to the DMSO control. In parallel, cell viability was assessed using the cell counting kit 8 assay in the absence of virus. Apilimod reduced viral transcript levels with an EC50 of 2.2 uM (FIG. 2A) which was below the half cytotoxic concentrations (CC50) of 20 uM (FIG. 2B) resulting in a selectivity index (CC50/EC50) of 9.1. Remdesivir was run in parallel and demonstrated an EC50 of 0.7 uM (FIG. 2C) and CC50 of 205 uM (FIG. 2D). The combination of 1 uM, 3 uM, or 10 uM apilimod and a suboptimal concentration of 0.5 uM remdesivir was also assessed. Apilimod displayed significantly greater inhibition in combination with remdesivir, than single agent remdesivir or single agent apilimod at all concentrations tested (FIG. 2E). The fold reduction in viral transcript versus the DMSO control from the same experiment was also determined (FIG. 2F). A significant dose dependent reduction in viral transcript was observed in combination with remdesivir versus single agents (6-2,180 fold). Together these data demonstrate that apilimod inhibits the infection of SARS-CoV-2 as single agent and displays highly significant activity of up to 2,180 fold reduction of viral transcripts in combination with remdesivir.

1. A method for treating or preventing a coronavirus infection in a subject in need thereof, the method comprising administering to the subject a composition comprising apilimod, or a pharmaceutically acceptable salt thereof.

2. The method of claim 1, further comprising administering at least one additional active agent selected from an additional PIKfyve inhibitor, a PD-L1 antagonist, a PD-L2 antagonist, and a PD-1 antagonist.

3. The method of claim 1, wherein the additional PIKfyve inhibitor is selected from the group consisting of APY0201, YM-201636, and pharmaceutically acceptable salts thereof.

4. The method of claim 1, wherein the apilimod is in the form of a free base or a dimesylate salt.

5. The method of claim 1, wherein the pharmaceutically acceptable salt is a monosalt selected from the group consisting of chloride, phosphate, maleate, L-tartrate, fumarate, DL lactate, and mesylate.

6. The method of claim 1, wherein the pharmaceutically acceptable salt is a disalt selected from the group consisting of mesylate, chloride, and bromide.

7. The method of claim 1, wherein the subject is a human.

8. The method of claim 1, further comprising administering to the subject an anti-viral agent.

9. The method of claim 8, wherein the anti-viral agent is one or more agents selected from the group consisting of interferon, remdesivir, azithromycin, hydroxychloroquine and chloroquine.

10. The method of claim 8, wherein the anti-viral agent is an interferon.

11. The method of claim 8, wherein the anti-viral agent is remdesivir.

12. The method of claim 8, wherein the anti-viral agent is azithromycin.

13. The method of claim 8, wherein the anti-viral agent is hydroxychloroquine.

14. The method of claim 8, wherein the anti-viral agent is chloroquine.

15. The method of claim 1, further comprising administering an anti-inflammatory agent.

16. The method of claim 13, wherein the anti-inflammatory agent is selected from tocilizumab and sarilumab.

17. The method of claim 1, wherein the subject in need is one who has symptoms of a respiratory viral infection including one or more of sore throat, nasal congestion and/or discharge, shortness of breath, difficulty breathing, and fever.

18. The method of claim 1 wherein the coronavirus is SARS-CoV-2.

19. The method of claim 16, wherein the method comprises administering apilimod, or a pharmaceutically acceptable salt thereof, in combination with one or more additional therapeutic agents selected from remdesivir, azithromycin, hydroxychloroquine, chloroquine, tocilizumab and sarilumab.

20. The method of claim 17, wherein the apilimod is administered in a separate dosage form, or in the same dosage form, as the one or more additional therapeutic agents.

21. A pharmaceutical pack or kit comprising, in separate containers or in a single container, a unit dose of apilimod, or a pharmaceutically acceptable salt thereof, and optionally, a unit dose of one or more of a PD-L1 antagonist, a PD-L2 antagonist, a PD-1 antagonist, and an anti-viral agent.

22. The pharmaceutical pack or kit of claim 19, wherein the anti-viral agent is an interferon.

23. A pharmaceutical pack or kit comprising, in separate containers or in a single container, a unit dose of apilimod, or a pharmaceutically acceptable salt thereof, and a unit dose of one or more additional therapeutic agents selected from remdesivir, azithromycin, hydroxychloroquine, chloroquine, tocilizumab and sarilumab.
